(19) United States
(12) Patent Application Publication
(10) Pub. No.: US 2003/0235565 A1
(43) Pub. Date: Dec. 25, 2003

(54) Feed additives for shrimp culture

(76) Inventor: Ling Yuk Cheung, Hong Kong (HK)

Correspondence Address: PENNIE AND EDMONDS, 1155 AVENUE OF THE AMERICAS, NEW YORK, NY, 100362711

(21) Appl. No.: 10/175,010

(22) Filed: Jun. 18, 2002

(51) Int. Cl. C12N 1/16; A01N 63/00

(52) U.S. Cl. 424/93.51; 435/254.21; 426/635

(57) ABSTRACT

The present invention relates to feed additives for shrimps in aquaculture. The invention provides methods for making a biological compositions comprising yeast cells that can improve the immune functions of shrimps in culture. The invention also relates to methods for manufacturing the biological compositions, and methods of using the biological compositions as feed additives.

### 1. FIELD OF THE INVENTION

The invention relates to biological compositions comprising yeast cells that can improve the immune functions of shrimps in culture. The invention also relates to methods for manufacturing the biological compositions, and methods of using the biological compositions as feed additives.

### 2. BACKGROUND OF THE INVENTION

Along with the rapid development of commercial aquaculture, there has been an accompanying increase in the occurrence of infectious and noninfectious diseases that reduce the yield. With increasing density and production level, outbreaks of fungal infections, e.g. Lagenidium and Sirolpidium; bacterial attacks, e.g. Vibrio and Aeromonas; and even viruses, e.g. Baculovirus, are not infrequent in hatcheries. The undesirable effects on the aquatic animals range from susceptibility to stress, reduced resistance to disease, to a slower growth rate. Many of the diseases are caused by organisms which are ubiquitous and have been found in major culture areas of the world, e.g. in Japan, Korea, Taiwan, the Philippines, Indonesia, Thailand, Malaysia, India, the Caribbean, Brazil, Mexico, Panama, Ecuador, Colombia, the U.S.A., and Australia.

Most of these problems are due to the absence of sanitary procedures such as those widely adopted in terrestrial husbandry, and insufficient control of the culturing systems, such as disinfection, regular dry-out, separate equipment for each tank, and separate rooms for maturation, spawning and hatching.

Although antifungal agents such as trifluralin and Malachite green, and antibiotics have achieved some success, the need to have a dry-out every six to eight weeks of production in order to eliminate bacterial strains which will become increasingly resistant is disruptive to the production process.

Antibiotics have been added to terrestrial animal feed since the 1940s. They are used to treat sick animals; to prevent other animals housed in confined barns or coops from infections; and to make the animals grow faster. Farmers give antibiotics, in low but daily doses, to entire herds or flocks to keep livestock healthy. The antibiotics also improve the absorption of nutrients, which helps the animals grow faster on less feed, and thus increase profits, particularly in intensive farming operations. However, the prophylactic use of antibiotics is unlikely to be practical in an aquaculture setting especially in open water facilities. More importantly, the use of antibiotics may exposes microorganisms to the antibiotics, thereby allowing antibiotic-resistant strains of the microorganisms to develop.

Because of concerns over the development of drug-resistance in microorganisms that cause human diseases, regulatory authorities in the United States and the European Union has banned or proposed banning the use of certain antibiotics in animal feed as a growth promoter. It is clear that while the scale and density of aquaculture increase, an urgent need for alternative means to reduce the incidence of infectious diseases in aquatic animals is emerging. The present invention provides a solution that uses specially treated yeasts to improve the immune functions of the aquatic animals.

The inclusion of yeast in aquaculture feed as a source of nutrient has been described. For examples, see:

U.S. Pat. No. 3,923,279 discloses a feed for aquatic animals that comprises marine or halophilic yeasts (Torulopisis, Rhodotorula, Saccharomyces species) which have been cultured in seawater containing waste molasses and an inorganic nitrogen compound.

U.S. Pat. No. 5,047,250 discloses a method of mixing baker's yeast with fish oil at up to 80° C. to form a feed substance that is suitable for direct feeding of fry, shellfish and mollusks.

U.S. Pat. No. 5,158,788 discloses a process for making aquaculture feed that is based on treating yeast cells with a chemical or enzyme that hydrolyses the external layer of the wall of the yeasts so as to improve its digestability to mollusks and crustaceans, and larvae thereof.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and does not constitute any admission as to the correctness of the dates or contents of these documents.

### 3. SUMMARY OF THE INVENTION

The present invention relates to biological compositions that can be added to aquaculture feed to reduce the incidence of infectious diseases in shrimps.

In one embodiment, the present invention provides biological compositions comprising a plurality of live yeast cells which are capable of improving the immune functions of shrimps and/or reducing the incidence of infectious diseases upon ingestion. In another embodiment, the invention provides methods of making the biological composition, and methods of using the biological composition as feed additive to maintain the health of shrimps in aquaculture.

In particular, the methods of the invention comprise culturing yeast cells in the presence of a series of electromagnetic fields such that the yeast cells becomes metabolically active and potent at stimulating an animal's immune system. Up to four different components of yeast cells can be used to form the biological compositions. Methods for manufacturing the biological compositions comprising culturing the yeast cells under activation conditions, mixing various yeast cell cultures of the present invention, followed by drying the yeast cells and packing the final product, are encompassed. In preferred embodiments, the starting yeast cells are commercially available and/or accessible to the public, such as but not limited to Saccharomyces cerevisiae.

The biological compositions of the invention can be fed directly to animals or used as an additive to be incorporated into regular animal feed. Animal feed compositions comprising activated yeast cells of the invention are encompassed by the invention.

### 4. BRIEF DESCRIPTION OF FIGURES

FIG. 1 Activation and conditioning of yeast cells. 1 yeast cell culture; 2 container; 3 electromagnetic field source; 4 electrode.

### 5. DETAILED DESCRIPTION OF THE INVENTION

The present invention relates to biological compositions that can be used to improve the immune functions of aquatic animals, and/or reduce the incidence of infectious diseases. The present invention provides methods for manufacturing the biological compositions as well as methods for using the biological compositions as aquaculture feed additives. Improved aquaculture feed comprising biological compositions of the invention are also encompassed.

The biological compositions of the invention comprise yeasts. Unlike the traditional use of yeasts as a component of the feed, the yeast cells of the invention are not a primary source of nutrients for the aquatic animals. The yeast cells of the invention serve as a supplement to replace or reduce the chemicals and antibiotics that are added to livestock feed. The yeast cells are live when administered orally or ingested along with feed by the aquatic animals. While in the gastrointestinal tract of an animal, the yeast cells are capable of stimulating the immune system and improving the immune functions of the animal, thereby reducing the incidence of infectious diseases. The use of the biological compositions of the invention can lower the overall cost of maintaining the health of animals in commercial aquaculture operations, and make feasible the minimal use or the elimination of chemicals and antibiotics in feed.

While the following terms are believed to have well-defined meanings in the art, the following are set forth to facilitate explanation of the invention.

As used herein, the term “feed” broadly refers to any kind of material, liquid or solid, that is used for nourishing an aquatic animal, and for sustaining normal or accelerated growth of an animal including larva, fry, and young developing animals.

The term “aquatic animal” as used herein refers to marine and freshwater shrimps, prawns and the like, and may include but not limited to the Penaeus species (Penaeid shrimps), Pendalus species (Pendalid shrimps), Macrobrachium species and Caridea shrimps. Examples may include kuruma prawn, Oriental shrimp, giant tiger shrimp, Oriental river prawn, offshore greasy back prawn, green tiger prawn, etc.

The term “immune functions” as used herein broadly encompasses specific and non-specific immunological reactions of the aquatic animal, and includes both humoral and cell-mediated defense mechanisms. The immune functions of the animal enable the animal to survive and/or recover from an infection by a pathogen, such as bacteria, viruses, fungi, protozoa, helminths, and other parasites. The immune functions of the animal can also prevent infections, particularly future infections by the same pathogen after the animal had an initial exposure to the pathogen. Many types of immune cells are involved in providing the immune functions, which include various subsets of hemocytes (non-granular, small granular, large granular). Details of the immune system of crustaceans are described in Invertebrate Blood: Cells and Serum Factors, Cheung (ed.), Perseus Books, 1984; and Invertebrate Immune Responses, Springer Verlag New York Incorporated, 1986, which are incorporated herein by reference in their entireties.

In one embodiment, the present invention provides biological compositions that comprise at least one yeast cell component. Each yeast cell component comprises a population of live yeast cells which have been cultured under a specific set of conditions such that the yeast cells are capable of improving the immune functions of aquatic animals. In preferred embodiments, the biological compositions of the invention comprise up to four yeast cell components.

According to the invention, under certain specific culture conditions, yeasts can be made metabolically active such that they become effective in stimulating and enhancing the immune functions of an animal which ingested the yeasts. Without being bound by any theory or mechanism, the inventor believes that the culture conditions activate and/or amplified the expression of a gene or a set of genes in the yeast cells such that the yeast cells becomes highly potent in stimulating the animal's immune system. It is envisioned that interactions between certain yeast gene products and elements of the animal's immune system is greatly enhanced by the elevated levels of these yeast gene products after the yeast cells have been cultured under the conditions described hereinbelow. The benefits of using the biological compositions are demonstrated by experimental results obtained from animals which show resistance to or rapid recovery from disease.

In one embodiment, the biological compositions of the invention can be fed directly to an animal. In another embodiment, the biological compositions can be added to the feed. As known to those skilled in the relevant art, many methods and appliances may be used to mix the biological compositions of the invention with feed. In a particular embodiment, a mixture of culture broths of the yeasts of the present invention are added directly to the feed just prior to feeding the animal. Dried powders of the yeasts can also be added directly to the feed just prior to feeding the animal. In yet another embodiment of the present invention, the yeast cells are mixed with the raw constituents of the feed with which the yeast cells become physically incorporated. The biological compositions may be applied to and/or mixed with the feed by any mechanized means which may be automated.

The amount of biological composition used depends in part on the feeding regimen and the type of feed, and can be determined empirically. For example, the useful ratio of biological composition to aquatic animal feed ranges from 0.1% to 1% by dry weight, preferably, 0.3 to 0.8%, and most preferably at about 0.5%. Although not necessary, the biological compositions of the invention can also be used in conjunction or in rotation with other types of supplements, such as but not limited to chemicals, vitamins, and minerals.

Described below in Section 5.1 and 5.2 are four yeast cell components of the invention and methods of their preparation. Section 5.3 describes the manufacture of the biological compositions of the invention which comprises at least one of the four yeast cell components.

5.1. Preparation of the Yeast Cell Cultures

The present invention provides yeast cells that are capable of improving the immune functions of an aquatic animal which ingested the yeast cells. Up to four different yeast cell components can be combined to make the biological compositions.

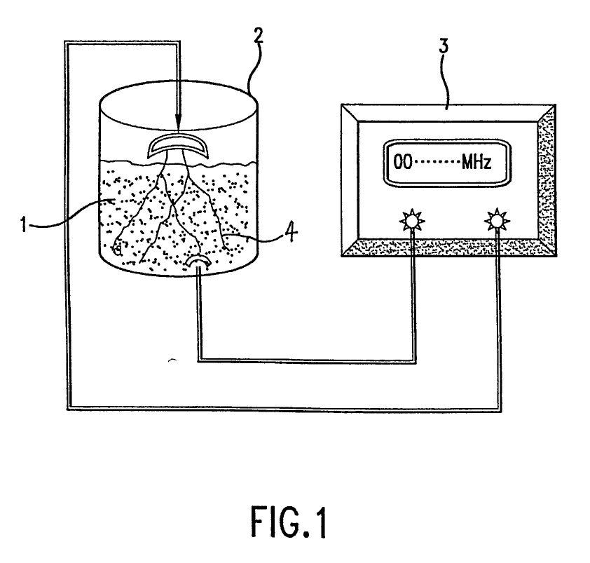

A yeast cell component of the biological composition is produced by culturing a plurality of yeast cells in an appropriate culture medium in the presence of an alternating electromagnetic field or multiple alternating electromagnetic fields in series over a period of time. The culturing process allows yeast spores to germinate, yeast cells to grow and divide, and can be performed as a batch process or a continuous process. As used herein, the terms “alternating electromagnetic field”, “electromagnetic field” or “EM field” are synonymous. An electromagnetic field useful in the invention can be generated by various means well known in the art. A schematic illustration of exemplary setups are depicted respectively in FIG. 1. An electromagnetic field of a desired frequency and a desired field strength is generated by an electromagnetic wave source (3) which comprises one or more signal generators that are capable of generating electromagnetic waves, preferably sinusoidal waves, and preferably in the frequency range of 1500 MHz-15000 MHz. Such signal generators are well known in the art. Signal generators capable of generating signal with a narrower frequency range can also be used. If desirable, a signal amplifier can also be used to increase the output signal, and thus the strength of the EM field.

The electromagnetic field can be applied to the culture by a variety of means including placing the yeast cells in close proximity to a signal emitter connected to a source of electromagnetic waves. In one embodiment, the electromagnetic field is applied by signal emitters in the form of electrodes that are submerged in a culture of yeast cells (1). In a preferred embodiment, one of the electrodes is a metal plate which is placed on the bottom of a non-conducting container (2), and the other electrode comprises a plurality of wires or tubes so configured inside the container such that the energy of the electromagnetic field can be evenly distributed in the culture. For an upright culture vessel, the tips of the wires or tubes are placed within 3 to 30 cm from the bottom of the vessel (i.e, approximately 2 to 10% of the height of the vessel from the bottom). The number of electrode wires used depends on both the volume of the culture and the diameter of the wire. For example, for a culture having a volume of 10 liter or less, two or three electrode wires having a diameter of between 0.5 to 2.0 mm can be used. For a culture volume of 10 liter to 100 liter of culture, the electrode wires or tubes can have a diameter of 3.0 to 5.0 mm. For a culture volume of 100 liter to 1000 liter, the electrode wires or tubes can have a diameter of 6.0 to 15.0 mm. For a culture having a volume greater than 1000 liter, the electrode wires or tubes can have a diameter of between 20.0 to 25.0 mm.

In various embodiments, yeasts of the genera of Saccharomyces, Candida, Crebrothecium, Geotrichum, Hansenula, Kloeckera, Lipomyces, Pichia, Rhodosporidium, Rhodotorula Torulopsis, Trichosporon, and Wickerhamia can be used in the invention.

Non-limiting examples of yeast strains include Saccharomyces cerevisiae Hansen, ACCC2034, ACCC2035, ACCC2036, ACCC2037, ACCC2038, ACCC2039, ACCC2040, ACCC2041, ACCC2042, AS2.1, AS2.4, AS2.11, AS2.14, AS2.16, AS2.56, AS2.69, AS2.70, AS2.93, AS2.98, AS2.101, AS2.109, AS2.110, AS2.112, AS2.139, AS2.173, AS2.174, AS2.182, AS2.196, AS2.242, AS2.336, AS2.346, AS2.369, AS2.374, AS2.375, AS2.379, AS2.380, AS2.382, AS2.390, AS2.393, AS2.395, AS2.396, AS2.397, AS2.398, AS2.399, AS2.400, AS2.406, AS2.408, AS2.409, AS2.413, AS2.414, AS2.415, AS2.416, AS2.422, AS2.423, AS2.430, AS2.431, AS2.432, AS2.451, AS2.452, AS2.453, AS2.458, AS2.460, AS2.463, AS2.467, AS2.486, AS2.501, AS2.502, AS2.503, AS2.504, AS2.516, AS2.535, AS2.536, AS2.558, AS2.560, AS2.561, AS2.562, AS2.576, AS2.593, AS2.594, AS2.614, AS2.620, AS2.628, AS2.631, AS2.666, AS2.982, AS2.1190, AS2.1364, AS2.1396, IFFI 1001, IFFI 1002, IFFI 1005, IFFI 1006, IFFI 1008, IFFI 1009, IFFI 1010, IFFI 1012, IFFI 1021, IFFI 1027, IFFI 1037, IFFI 1042, IFFI 1043, IFFI 1045, IFFI 1048, IFFI 1049, IFFI 1050, IFFI 1052, IFFI 1059, IFFI 1060, IFFI 1063, IFFI 1202, IFFI 1203, IFFI 1206, IFFI 1209, IFFI 1210, IFFI 1211, IFFI 1212, IFFI 1213, IFFI 1215, IFFI 1220, IFFI 1221, IFFI 1224, IFFI 1247, IFFI 1248, IFFI 1251, IFFI 1270, IFFI 1277, IFFI 1287, IFFI 1289, IFFI 1290, IFFI 1291, IFFI 1292, IFFI 1293, IFFI 1297, IFFI 1300, IFFI 1301, IFFI 1302, IFFI 1307, IFFI 1308, IFFI 1309, IFFI 1310, IFFI 1311, IFFI 1331, IFFI 1335, IFFI 1336, IFFI 1337, IFFI 1338, IFFI 1339, IFFI 1340, IFFI 1345, IFFI 1348, IFFI 1396, IFFI 1397, IFFI 1399, IFFI 1411, IFFI 1413; Saccharomyces cerevisiae Hansen Var. ellipsoideus (Hansen) Dekker, ACCC2043, AS2.2, AS2.3, AS2.8, AS2.53, AS2.163, AS2.168, AS2.483, AS2.541, AS2.559, AS2.606, AS2.607, AS2.611, AS2.612; Saccharomyces chevalieri Guillermond, AS2.131, AS2.213; Saccharomyces delbrueckii, AS2.285; Saccharomyces delbrueckii Lindner var. mongolicus Lodder et van Rij, AS2.209, AS2.1157; Saccharomyces exiguous Hansen, AS2.349, AS2.1158; Saccharomyces fermentati (Saito) Lodder et van Rij, AS2.286, AS2.343; Saccharomyces logos van laer et 30 Denamur ex Jorgensen, AS2.156, AS2.327, AS2.335; Saccharomyces mellis Lodder et Kreger Van Rij, AS2.195; Saccharomyces microellipsoides Osterwalder, AS2.699; Saccharomyces oviformis Osterwalder, AS2.100; Saccharomyces rosei (Guilliermond) Lodder et kreger van Rij, AS2.287; Saccharomyces rouxii Boutroux, AS2.178, AS2.180, AS2.370, AS2.371; Saccharomyces sake Yabe, ACCC2045; Candida arborea, AS2.566; Candida Krusei (Castellani) Berkhout, AS2.1045; Candida lambica (Lindner et Genoud) van.Uden et Buckley, AS2.1182; Candida lipolytica (Harrison) Diddens et Lodder, AS2.1207, AS2.1216, AS2.1220, AS2.1379, AS2.1398, AS2.1399, AS2.1400; Candida parapsilosis (Ashford) Langeron et Talice, AS2.590; Candida parapsilosis (Ashford) et Talice Var. intermedia Van Rij et Verona, AS2.491; Candida pulcherriman (Lindner) Windisch, AS2.492; Candida rugousa (Anderson) Diddens et Loddeer, AS2.511, AS2.1367, AS2.1369, AS2.1372, AS2.1373, AS2.1377, AS2.1378, AS2.1384; Candida tropicalis (Castellani) Berkout, ACCC2004, ACCC2005, ACCC2006, AS2.164, AS2.402, AS2.564, AS2.565, AS2.567, AS2.568, AS2.617, AS2.1387; Candida utilis Henneberg Lodder et Kreger Van Rij, AS2.120, AS2.281, AS2.1180; Crebrothecium ashbyii (Guillermond) Routein, AS2.481, AS2.482, AS2.1197; Geotrichum candidum Link, ACCC2016, AS2.361, AS2.498, AS2.616, AS2.1035, AS2.1062, AS2.1080, AS2.1132, AS2.1175, AS2.1183; Hansenula anomala (Hansen) H et P sydow, ACCC2018, AS2.294, AS2.295, AS2.296, AS2.297, AS2.298, AS2.299, AS2.300, AS2.302, AS2.338, AS2.339, AS2.340, AS2.341, AS2.470, AS2.592, AS2.641, AS2.642, AS2.635, AS2.782, AS2.794; Hansenula arabitolgens Fang, AS2.887; Hansenula jadinii Wickerham, ACCC2019; Hansenula saturnus (Klocker) H et P sydow, ACCC2020; Hansenula schneggii (Weber) Dekker, AS2.304; Hansenula subpelliculosa Bedford, AS2.738, AS2.740, AS2.760, AS2.761, AS2.770, AS2.783, AS2.790, AS2.798, AS2.866; Kloeckera apiculata (Reess emend. Klocker) Janke, ACCC2021, ACCC2022, ACCC2023, AS2.197, AS2.496, AS2.711, AS2.714; Lipomyces starkeyi Lodder et van Rij, ACCC2024, AS2.1390; Pichia farinosa (Lindner) Hansen, ACCC2025, ACCC2026, AS2.86, AS2.87, AS2.705, AS2.803; Pichia membranaefaciens Hansen, ACCC2027, AS2.89, AS2.661, AS2.1039; Rhodosporidium toruloides Banno, ACCC2028; Rhodotorula glutinis (Fresenius) Harrison, ACCC2029, AS2.280, ACCC2030, AS2.102, AS2.107, AS2.278, AS2.499, AS2.694, AS2.703, AS2.704, AS2.1146; Rhodotorula minuta (Saito) Harrison, AS2.277; Rhodotorula rubar (Demme) Lodder, ACCC2031, AS2.21, AS2.22, AS2.103, AS2.105, AS2.108, AS2.140, AS2.166, AS2.167, AS2.272, AS2.279, AS2.282; Saccharomyces carlsbergensis Hansen, AS2.113, ACCC2032, ACCC2033, AS2.312, AS2.116, AS2.118, AS2.121, AS2.132, AS2.162, AS2.189, AS2.200, AS2.216, AS2.265, AS2.377, AS2.417, AS2.420, AS2.440, AS2.441, AS2.443, AS2.444, AS2.459, AS2.595, AS2.605, AS2.638, AS2.742, AS2.745, AS2.748, AS2.1042; Saccharomyces uvarum Beijer, IFFI 1023, IFFI 1032, IFFI 1036, IFFI 1044, IFFI 1072, IFFI 1205, IFFI 1207; Saccharomyces willianus Saccardo, AS2.5, AS2.7, AS2.119, AS2.152, AS2.293, AS2.381, AS2.392, AS2.434, AS2.614, AS2.1189; Saccharomyces sp., AS2.311; Saccharomyces ludwigii Hansen, ACCC2044, AS2.243, AS2.508; Saccharomyces sinenses Yue, AS2.1395; Schizosaccharomyces octosporus Beijerinck, ACCC 2046, AS2.1148; Schizosaccharomyces pombe Linder, ACCC2047, ACCC2048, AS2.248, AS2.249, AS2.255, AS2.257, AS2.259, AS2.260, AS2.274, AS2.994, AS2.1043, AS2.1149, AS2.1178, IFFI 1056; Sporobolomyces roseus Kluyver et van Niel, ACCC 2049, ACCC 2050, AS2.619, AS2.962, AS2.1036, ACCC2051, AS2.261, AS2.262; Torulopsis candida (Saito) Lodder, ACCC2052, AS2.270; Torulopsis famta (Harrison) Lodder et van Rij, ACCC2053, AS2.685; Torulopsis globosa (Olson et Hammner) Lodder et van Rij, ACCC2054, AS2.202; Torulopsis inconspicua Lodder et van Rij, AS2.75; Trichosporon behrendii Lodder et Kreger van Rij, ACCC2055, AS2.1193; Trichosporon capitatum Diddens et Lodder, ACCC2056, AS2.1385; Trichosporon cutaneum (de Beurm et al.)Ota, ACCC2057, AS2.25, AS2.570, AS2.571, AS2.1374; Wickerhamia fluoresens (Soneda) Soneda, ACCC2058, AS2.1388. Yeasts of the Saccharomyces genus are generally preferred. Among strains of Saccharomyces cerevisiae, Saccharomyces cerevisiae Hansen is a preferred strain.

Generally, yeast strains useful for the invention can be obtained from private or public laboratory cultures, or publically accessible culture deposits, such as the American Type Culture Collection, 10801 University Boulevard, Manassas, Va. 20110-2209 and the China General Microbiological Culture Collection Center (CGMCC), China Committee for Culture Collection of Microorganisms, Institute of Microbiology, Chinese Academy of Sciences, Haidian, P.O. Box 2714, Beijing, 100080, China.

Although it is preferred, the preparation of the yeast cell components of the invention is not limited to starting with a pure strain of yeast. Each yeast cell component may be produced by culturing a mixture of yeast cells of different species or strains. The constituents of a yeast cell component can be determined by standard yeast identification techniques well known in the art.

In various embodiments of the invention, standard techniques for handling, transferring, and storing yeasts are used. Although it is not necessary, sterile conditions or clean environments are desirable when carrying out the manufacturing processes of the invention. Standard techniques for handling animal body fluids and immune cells, and for studying immune functions of an animal are also used. Details of such techniques are described in Current Protocols In Immunology, 1991, Coligan, et al. (Ed), John Wiley & Sons, Inc., which is incorporated herein by reference in its entirety.

In one embodiment, the yeast cells of the first yeast cell component are cultured in the presence of at least one alternating electromagnetic (EM) field with a frequency in the range of 7497 MHz to 7516 MHz. A single EM field or a series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 7497, 7498, 7499, 7500, 7501, 7502, 7503, 7504, 7505, 7506, 7507, 7508, 7509, 7510, 7511, 7512, 7513, 7514, 7515, or 7516 MHz.

The field strength of the EM field(s) is in the range of 3.5 to 200 mV/cm. In a preferred embodiment, the EM field(s) at the beginning of a series have a lower EM field strength than later EM field(s), such that the yeast cell culture are exposed to EM fields of progressively increasing field strength. Accordingly, the yeast cells can be cultured at the lower EM field strength (e.g., 50-80 mV/cm) for 26 to 70 hours and then cultured at the higher EM field strength (e.g., 100-180 mV/cm) for another 28 to 70 hours. The yeast culture can remain in the same container and use the same set of electromagnetic wave generator and emitters when switching from one EM field to another EM field.

The culture process can be initiated by inoculating 100 ml of medium with 1 ml of an inoculum of the selected yeast strain(s) at a cell density of about 10⁵ cells/ml. The starting culture is kept at 25° C. to 35° C. preferably 28° to 32° C. for 24 to 48 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 1 provides an exemplary medium for culturing the first yeast cell component of the invention.

TABLE 1

| Medium Composition | Quantity | |
|---|---|---|
| Sucrose or glucose | 20.0 | g |
| K₂HPO₄ | 0.25 | g |
| MgSO₄.7H₂O | 0.2 | g |
| NaCl | 0.22 | g |
| CaSO₄.2H₂O | 0.5 | g |
| CaCO₃ | 6.0 | g |
| Urea | 0.2 to 5.0 | g |
| Body fluid of the animals | 2-5 | ml |
| Autoclaved water | 1000 | ml |
| Agar (if a solid medium is desired) | 10-20 | g |

It should be noted that the composition of the media provided in Table 1 is not intended to be limiting. The process can be scaled up or down according to needs. Various modifications of the culture medium may be made by those skilled in the art, in view of practical and economic considerations, such as the scale of culture and local supply of media components.

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCi, and CaSO₄.

The body fluid of the animals is obtained by scraping the surface of the shrimp body with a firm edge, such as a blade made of bamboo, and collecting the secretions produced by the shrimp body. From 500 to 2000 freshwater shrimps each weighing 5-10 g, about 10 to 20 ml of the body fluid can be collected. The same technique is used to collect the body fluid of marine shrimps. Using 500-1000 marine shrimps each weighing 10-100 g, about 10-20 ml of the body fluid can be obtained. The body fluids of the shrimps comprise hemocytes, and can be mixed, diluted, or concentrated before use.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the first yeast cell component of the invention may be recovered from the culture by various methods known in the art, and stored at a temperature below about 0° C. to 4° C. The recovered yeast cells may also be dried and stored in powder form.

A non-limiting example of making a first yeast cell component of the invention with Saccharomyces cerevisiae strain AS2.502 is provided in Section 6 hereinbelow.

In another embodiment, the yeast cells of the second yeast cell component are cultured in the presence of at least one alternating electromagnetic (EM) field with a frequency in the range of 6800 MHz to 6820 MHz. A single EM field or a series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 6800, 6801, 6802, 6803, 6804, 6805, 6806, 6807, 6808, 6809, 6810, 6811, 6812, 6813, 6814, 6815, 6816, 6817, 6818, 6819, or 6820 MHz.

The field strength of the EM field(s) is in the range of 4.5 to 190 mV/cm. In a preferred embodiment, the EM field(s) at the beginning of a series have a lower EM field strength than later EM field(s), such that the yeast cell culture are exposed to EM fields of progressively increasing field strength. Accordingly, the yeast cells can be cultured at the lower EM field strength (e.g., 50-80 mV/cm) for 20 to 60 hours and then cultured at the higher EM field strength (e.g., 120-190 mV/cm) for another 20 to 60 hours. The yeast culture can remain in the same container and use the same set of electromagnetic wave generator and emitters when switching from one EM field to another EM field.

The culture process can be initiated by inoculating 100 ml of medium with 1 ml of an inoculum of the selected yeast strain(s) at a cell density of about 10⁵ cells/ml. The starting culture is kept at 25° C. to 35° C. for 24 to 48 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 2 provides an exemplary medium for culturing the second yeast cell component of the invention.

TABLE 2

| Medium Composition | Quantity | |
|---|---|---|
| Sucrose or soluble starch | 20.0 | g |
| K₂HPO₄ | 0.25 | g |
| MgSO₄.7H₂O | 0.2 | g |
| NaCl | 0.22 | g |
| CaCO₃ | 0.5 | g |
| Urea | 2.0 | g |
| Body fluid of the animals | 2-5 | ml |
| Autoclaved water | 1000 | ml |
| Agar (if a solid medium is desired) | 15 | g |

It should be noted that the composition of the media provided in Table 2 is not intended to be limiting. The process can be scaled up or down according to needs. Various modifications of the culture medium may be made by those skilled in the art, in view of practical and economic considerations, such as the scale of culture and local supply of media components.

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCl, and CaSO₄. Body fluid of the animals can be obtained by the method as described before.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the second yeast cell component of the invention may be recovered from the culture by various methods known in the art, and stored at a temperature below about 0° C. to 4° C. The recovered yeast cells may also be dried and stored in powder form.

A non-limiting example of making a second yeast cell component of the invention with Saccharomyces cerevisiae strain AS2.562 is provided in Section 6 hereinbelow.

In yet another embodiment, the yeast cells of the third yeast cell component are cultured in the presence of at least one alternating electromagnetic (EM) field with a frequency in the range of 8300 MHz to 8320 MHz. A single EM field or a series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 8300, 8301, 8302, 8303, 8304, 8305, 8306, 8307, 8308, 8309, 8310, 8311, 8312, 8313, 8314, 8315, 8316, 8317, 8318, 8319, or 8320 MHz.

The field strength of the EM field(s) is in the range of 11 to 290 mV/cm. In a preferred embodiment, the EM field(s) at the beginning of a series have a lower EM field strength than later EM field(s), such that the yeast cell culture are exposed to EM fields of progressively increasing field strength. Accordingly, the yeast cells can be cultured at the lower EM field strength (e.g., 120-150 mV/cm) for 26 to 60 hours and then cultured at the higher EM field strength (e.g., 200-290 mV/cm) for another 25 to 60 hours. The yeast culture can remain in the same container and use the same set of electromagnetic wave generator and emitters when switching from one EM field to another EM field.

The culture process can be initiated by inoculating 100 ml of medium with 1 ml of an inoculum of the selected yeast strain(s) at a cell density of about 10⁵ cells/ml. The starting culture is kept at 25° C. to 35° C. for 24 to 48 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 3 provides an exemplary medium for culturing the third yeast cell component of the invention.

TABLE 3

| Medium Composition | Quantity | |
|---|---|---|
| Sucrose or soluble starch | 20.0 | g |
| MgSO₄.7H₂O | 0.25 | g |
| NaCl | 0.2 | g |
| Ca(H₂PO₄) | 0.22 | g |
| CaCO₃ | 0.5 | g |
| (NH₄)₂HPO₄ | 2.0 | g |
| K₂HPO₄ | 0.3 | g |
| Body fluid of the animals | 2-5 | ml |
| Autoclaved water | 1000 | ml |
| Agar (if a solid medium is desired) | 15 | g |

It should be noted that the composition of the media provided in Table 3 is not intended to be limiting. The process can be scaled up or down according to needs. Various modifications of the culture medium may be made by those skilled in the art, in view of practical and economic considerations, such as the scale of culture and local supply of media components.

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCl, and CaSO₄. Body fluid of the animals can be obtained by the method as described before.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the third yeast cell component of the invention may be recovered from the culture by various methods known in the art, and stored at a temperature below about 0° C. to 4° C. The recovered yeast cells may also be dried and stored in powder form.

A non-limiting example of making a third yeast cell component of the invention with Saccharomyces cerevisiae strain AS2.982 is provided in Section 6 hereinbelow.

In yet another embodiment, the yeast cells of the fourth yeast cell component are cultured in the presence of at least one alternating electromagnetic (EM) field with a frequency in the range of 8425 MHz to 8445 MHz. A single EM field or a series of EM fields can be applied, each having a different frequency within the stated range, or a different field strength within the stated range, or different frequency and field strength within the stated ranges. Although any practical number of EM fields can be used within a series, it is preferred that, the yeast culture be exposed to a total of 2, 3, 4, 5, 6, 7, 8, 9 or 10 different EM fields in a series. The EM field(s), which can be applied by any means known in the art, can each have a frequency of 8425, 8426, 8427, 8428, 8429, 8430, 8431, 8432, 8433, 8434, 8435, 8436, 8437, 8438, 8439, 8440, 8441, 8442, 8443, 8444, or 8445 MHz.

The field strength of the EM field(s) is in the range of 14 to 320 mV/cm. In a preferred embodiment, the EM field(s) at the beginning of a series have a lower EM field strength than later EM field(s), such that the yeast cell culture are exposed to EM fields of progressively increasing field strength. Accordingly, the yeast cells can be cultured at the lower EM field strength (e.g., 150-180 mV/cm) for 25 to 70 hours and then cultured at the higher EM field strength (e.g., 180-320 mV/cm) for another 25 to 70 hours. The yeast culture can remain in the same container and use the same set of electromagnetic wave generator and emitters when switching from one EM field to another EM field.

The culture process can be initiated by inoculating 100 ml of medium with 1 ml of an inoculum of the selected yeast strain(s) at a cell density of about 10⁵ cells/ml. The starting culture is kept at 25° C. to 35° C. for 24 to 48 hours prior to exposure to the EM field(s). The culturing process may preferably be conducted under conditions in which the concentration of dissolved oxygen is between 0.025 to 0.08 mol/m³, preferably 0.04 mol/m³. The oxygen level can be controlled by any conventional means known in the art, including but not limited to stirring and/or bubbling.

The culture is most preferably carried out in a liquid medium which contains animal serum and sources of nutrients assimilable by the yeast cells. Table 4 provides an exemplary medium for culturing the fourth yeast cell component of the invention.

TABLE 4

| Medium Composition | Quantity | |
|---|---|---|
| Starch | 20.0 | g |
| (NH₄)₂HPO₄ | 0.25 | g |
| K₂HPO₄ | 0.2 | g |
| MgSO₄.7H₂O | 0.22 | g |
| NaCl | 0.5 | g |
| CaSO₄.2H₂O | 0.3 | g |
| CaCO₃ | 3.0 | g |
| Body fluid of the animals | 2-5 | ml |
| Autoclaved water | 1000 | ml |
| Agar (if a solid medium is desired) | 15 | g |

It should be noted that the composition of the media provided in Table 4 is not intended to be limiting. The process can be scaled up or down according to needs. Various modifications of the culture medium may be made by those skilled in the art, in view of practical and economic considerations, such as the scale of culture and local supply of media components.

In general, carbohydrates such as sugars, for example, sucrose, glucose, fructose, dextrose, maltose, xylose, and the like and starches, can be used either alone or in combination as sources of assimilable carbon in the culture medium. The exact quantity of the carbohydrate source or sources utilized in the medium depends in part upon the other ingredients of the medium but, in general, the amount of carbohydrate usually varies between about 0.1% and 5% by weight of the medium and preferably between about 0.5% and 2%, and most preferably about 0.8%. These carbon sources can be used individually, or several such carbon sources may be combined in the medium. Among the inorganic salts which can be incorporated in the culture media are the customary salts capable of yielding sodium, calcium, phosphate, sulfate, carbonate, and like ions. Non-limiting examples of nutrient inorganic salts are (NH₄)₂HPO₄, CaCO₃, MgSO₄, NaCl, and CaSO₄. Body fluid of the animals can be obtained by the method as described before.

Although the yeast cells will become activated even after a few hours of culturing in the presence of the EM field(s), the yeast cells can be cultured in the presence of the EM field(s) for an extended period of time (e.g., one or more weeks). At the end of the culturing process, the yeast cells which constitute the forth yeast cell component of the invention may be recovered from the culture by various methods known in the art, and stored at a temperature below about 0° C. to 4° C. The recovered yeast cells may also be dried and stored in powder form.

A non-limiting example of making a fourth yeast cell component of the invention with Saccharomyces cerevisiae strain AS2.406 is provided in Section 6 hereinbelow.

5.2. Conditioning of the Yeast Cells

In another aspect of the invention, the performance of the activated yeast cells can be optimized by culturing all the activated yeast cells in the presence of materials taken from the gastrointestinal tract of the type of animal to which the biological composition will be fed. The inclusion of this additional conditioning process allows the activated yeast cells to adapt to and endure the acidic environment of the animal's stomach.

According to the invention, activated yeast cells prepared as described in Section 5.1 can be further cultured as a mixture in a medium with a composition as shown in Table 5.

TABLE 5
(Per 1000 ml of culture medium)

| Medium Composition | Quantity | |
|---|---|---|
| Extracts from the digestive tracts of | 300 | ml; stored at 4° C. |
| shrimps | | |
| Wild jujube juice | 300 | ml |
| Wild hawthorn juice | 320 | ml |
| (NH₄)₂HPO₄ | 0.25 | g |
| K₂HPO₄ | 0.2 | g |
| MgSO₄.7H₂O | 0.22 | g |
| NaCl | 0.5 | g |
| CaSO₄.2H₂O | 0.3 | g |
| CaCO₃ | 3.0 | g |
| Yeast cell culture (up to 4 different | 20 | ml each |
| cultures, containing 1 × 10⁸/ml) | | |

The process can be scaled up or down according to needs.

The wild jujube juice is a filtered extract of wild jujube fruits prepared by mixing 5 ml of water per gram of crushed wild jujube. The wild hawthorn juice is a filtered extract of wild hawthorn fruits prepared by mixing 5 ml of water per gram of crushed wild hawthorn.

Extracts from the digestive tracts of shrimps can be prepared by removing the digestive tract from the shrimp body, collecting the contents of the digestive tract and mixing it with distilled water. Up to 10 to 30 g of the contents of shrimp digestive tracts can be obtained from 500 to 1000 shrimps each weighing 10 to 20 g, and mixed with 1000 to 2000 ml. The mixture is filtered and stored at 4° C. or −20 ° C.

The mixture of yeast cells is cultured for about 48 to 96 hours in the presence of a series of electromagnetic fields. Each electromagnetic field has a frequency that, depending on the strains of yeast included, corresponds to one of the four ranges of frequencies described in Sections 5.1. If all four yeast components are present, a combination of the following four frequency bands can be used: 7497-7516 MHz; 6800-6820 MHz; 8300-8320 MHz; 8425-8445 MHz. Generally, the yeast cells are subjected to an EM field strength in the range from 85 mV/cm to 320 mV/cm in this process. The EM fields in the four frequency bands can be applied simultaneously or sequentially.

While the yeast cell culture is exposed to the EM field(s), the culture is incubated at temperatures that cycle between about 5° C. to about 35° C. For example, in a typical cycle, the temperature of the culture may start at about 35° C. and be allowed to fall gradually to about 5° C., and then gradually be brought up to about 35° C. for another cycle. Each complete cycle lasts about 3 hours. The cycles are repeated until the yeast cells are recovered. The recovered yeast cells can be stored under 4° C.

5.3 Manufacture of the Biological Compositions

The present invention further provides a method for manufacturing a biological composition that comprises the yeast cells of the invention. Preferably, the biological compositions of the invention comprise yeast cells activated by the methods described in section 5.1 and which have been subject to adaptive culturing or conditioning as described in section 5.2. Most preferably, the biological compositions comprise all four yeast cell components.

To mass produce the biological compositions of the invention, the culture process is scaled up accordingly. To illustrate the scaled-up process, a method for producing 1000 kg of the biological composition is described as follows:

For each of the four yeast cell components, a 1000 ml stock culture of the activated and conditioned yeast cells (about 1×10¹⁰ cells/ml) is used to inoculate a culture comprising 100 kg starch, 250 liters of clean water (at 20° C. to 45° C.) and the ingredients used in the activation of the yeast cells (about 20-40 g of culture media components as described in Table 1, 2, 3, or 4). The four 250-liter cultures containing the four yeast cell components are then combined and cultured at 35° to 37° C. in the presence of an EM field(s) of the four ranges of frequencies as described in section 5.1, and a field strength of between 120 to 450 mV/cm. The EM fields may be applied simultaneously or sequentially. The culture process is carried out for about 48 to 96 hours, or when the yeast cell number reaches a density of greater than about 2×10⁹ cells/ml. At this point, the yeast cells must be stored at about 15° to 20° C., and if not used immediately, dried for storage within 24 hours.

The prepared yeast cells and biological compositions can be dried in a two-stage drying process. During the first drying stage, the yeast cells are dried in a first dryer at a temperature not exceeding 65° C. for a period of time not exceeding 10 minutes so that yeast cells quickly become dormant. The yeast cells are then sent to a second dryer and dried at a temperature not exceeding 70° C. for a period of time not exceeding 30 minutes to further remove water. After the two stages, the water content should be lower than 5%. It is preferred that the temperatures and drying times be adhered to in both drying stages so that yeast cells do not lose their vitality and functions. The dried yeast cells are then cooled to room temperature. The dried yeast cells may also be screened in a separator so that particles of a preferred size are selected. The dried cells can then be sent to a bulk bag filler for packing.

### 6. EXAMPLE

The following example illustrates the manufacture of a biological composition that can be used as an animal feed additive.

The biological composition comprises the following four components of yeasts: Saccharomyces cerevisiae AS2.502, AS2.562, AS2.982 and AS2.406. Each of the yeast components is capable of increasing the growth rate and/or health of shrimps in aquaculture resulting in a gain in overall body weight of shrimps in aquaculture. The four yeast cell components are prepared separately as follows:

A starting culture containing about 10⁵ cells/ml of AS2.502 is placed into the container (2) as shown in FIG. 1 containing a medium with the composition as shown in Table 1. Initially, the yeast cells are cultured for about 33 hours at 28° C. without an EM field. Then, in the same medium, at 28° C., the yeast cells are cultured in the presence of a series of eight EM fields applied in the order stated: 7500 MHz at 68 mV/cm for 8 hrs; 7507 MHz at 68 mV/cm for 8 hrs; 7511 MHz at 68 mV/cm for 26 hrs; 7515 MHz at 68 mV/cm for 26 hrs; 7500 MHz at 176 mV/cm for 8 hrs; 7507 MHz at 176 mV/cm for 8 hrs; 7511 MHz at 176 mV/cm for 28 hrs; 7515 MHz at 176 mV/cm for 28 hrs. The yeast cells were conditioned by further culturing shrimps in an extract from the digestive tracts of shrimps juice, jujube juice and hawthorn juice as described in section 5.2, in the presence of a series of two EM fields: 7511 MHz at 176 mV/cm for 28 hours and 7515 MHz at 176 mV/cm for 28 hours. After the last culture period, the yeast cells are either used within 24 hours to make the biological compositions, or dried for storage as described in section 5.3.

The beneficial effect of this first component of yeast cells on animals was tested as follows: The test was conducted with a species of Chinese shrimp, all having a body length of 1.6±0.1 cm. Four tanks were used per test while the total starting weight of shrimp in each tank is within ≦1%. The test was repeated three times, thus involving a total of 12 tanks. The first group of animals (Group A) were fed a diet comprising a mixture of antibiotics as shown in Table 6A, and cultured in water that has been treated with the chemicals in Table 6B.

TABLE 6A
composition of animal feed containing antibiotics

| | Quantities per metric | | |
|---|---|---|---|
| Ingredients | ton | | Notes |
| basic feed | 100 | kg | Does not contain antibiotics; |
| | | | supplied by Shandong Halobios |
| | | | Cultivate Institute |
| sulfadiazine | 60 | g/ton | |
| streptomycin | 100 | g/t | 10⁷ IU |
| erythromycin | 100 | g/t | 10⁷ IU |
| chloromycetin | 100 | g/t | 10⁷ IU |
| oxytetracycline | 100 | g/t | 10⁷ IU |
| sulfaguanidine | 60 | g/t | |
| furacilin | 70 | g/t | |
| furazolidone | 50 | g/t | |
| (furoxone) | | | |

TABLE 6B
Traditional chemicals used to clean the water environment.

| Ingredient | Quantities in g/m³ water | | Notes |
|---|---|---|---|
| calcium oxide | 40 | g/m³ | used monthly |
| bleaching powder | 20 | g/m³ | used monthly |
| copper sulfate | 2.0 | g/m³ | used twice monthly |
| ferrous sulfate | 2.0 | g/m³ | used twice monthly |
| trichlorphon | 0.2 | g/m³ | used monthly |
| furacilin | 70 | g/t | used monthly |
| furazolidone (furoxone) | 50 | g/t | used monthly |

The animals of Group B were fed a diet comprising activated AS2.502 yeast cells. The activated yeast cells were present in an additive which was prepared by mixing dried cells with zeolite powder (less than 200 mesh) at a ratio of about 10⁹ to 10¹⁰ yeast cells per gram of zeolite powder. For every 995 kg of basic feed, 5 kg of the additive was added, yielding an additive that comprises 0.5% yeast additive by weight, i.e, there is about 5×10¹² to 5×10¹³ yeast cells in 1000 kg of feed with additive. The third group of animals (Group C) was fed a diet which contains an additive that was prepared identically to that used in Group B except that the AS2.502 yeast cells were not activated. The animals of Group D were fed the basic diet with neither antibiotic nor yeast additives. None of the tanks and water in Group B, C, or D were treated with the chemicals in Table 6B. After five months, the health status of the animals in various groups are shown in Table 7 below.

TABLE 7
Yield of aquatic animals fed with different diets

| Group | Total weight of 3 tanks | % relative to Group A |
|---|---|---|
| A | 63.5 kg | 100 |
| B | 70.2 kg | 110.5 |
| C | 27.6 kg | 43.5 |
| D | 25.3 kg | 39.8 |

To prepare the second component, a starting culture containing about 10⁵ cells/ml of AS2.562 is placed into the container (2) as shown in FIG. 1 containing a medium with the composition as shown in Table 2. Initially, the yeast cells are cultured for about 32 hours at 31° C. without an EM field. Then, in the same medium, at 31° C., the yeast cells are cultured in the presence of a series of eight EM fields applied in the order stated: 6801 MHz at 75 mV/cm for 18 hrs; 6807 MHz at 75 mV/cm for 18 hrs; 6810 at 75 mV/cm for 11 hrs; 6815 MHz at 75 mV/cm for 11 hrs; 6801 MHz at 185 mV/cm for 21 hrs; 6807 MHz 185 mV/cm for 21 hrs; 6810 MHz 185 mV/cm for 9 hrs; 6815 MHz 185 mV/cm for 9 hrs. The yeast cells were conditioned by further culturing in an extract from the digestive tracts of shrimps, jujube juice and hawthorn juice as described in section 5.2, in the presence of a series of two EM fields: 6801 MHz at 185 mV/cm for 21 hours and 6807 MHz at 185 mV/cm for 21 hours. After the last culture period, the yeast cells are either used within 24 hours to make the biological compositions, or dried for storage as described in section 5.3.

The beneficial effect of this second component of yeast cells on animals was tested as follows: The test was conducted with a species of Chinese shrimp, all having a body length of 1.6±0.1 cm. Four tanks were used per test while the total starting weight of shrimp in each tank is within ≦1%. The test was repeated three times, thus involving a total of 12 tanks. The first group of animals (Group A) were fed a diet comprising a mixture of antibiotics as shown in Table 6A, and cultured in water that has been treated with the chemicals in Table 6B.

The animals of Group B were fed a diet comprising activated AS2.562 yeast cells. The activated yeast cells were present in an additive which was prepared by mixing dried cells with zeolite powder (less than 200 mesh) at a ratio of 1×10⁹ yeast cells per gram of zeolite powder. For every 995 kg of basic feed, 5 kg of the additive was added, yielding an additive that comprises 0.5% yeast additive by weight. The third group of animals (Group C) was fed a diet which contains an additive that was prepared identically to that used in Group B except that the AS2.562 yeast cells were not activated. The animals of Group D were fed the basic diet with neither antibiotic nor yeast additives. After five months, the health status of the animals in various groups are shown in Table 8 below.

TABLE 8
Yield of aquatic animals fed with different diets

| Group | Total weight of 3 tanks | % relative to Group A |
|---|---|---|
| A | 65.6 kg | 100 |
| B | 70.9 kg | 108.1 |
| C | 28.1 kg | 42.8 |
| D | 25.1 kg | 38.3 |

For the third yeast cell component, a starting culture containing about 10⁵ cells/ml of AS2.982 is placed into the container (2) as shown in FIG. 1 containing a medium with the composition as shown in Table 3. Initially, the yeast cells are cultured for about 26 hours at 29° C. without an EM field. Then, in the same medium, at 29° C., the yeast cells are cultured in the presence of a series of eight EM fields applied in the order stated: 8301 MHz at 135 mV/cm for 26 hrs; 8307 MHz at 135 mV/cm for 26 hrs; 8311 MHz at 135 mV/cm for 10 hrs; 8318 MHz at 135 mV/cm for 10 hrs; 8301 MHz at 256 mV/cm for 15 hrs; 8307 MHz at 256 mV/cm for 15 hrs; 8311 MHz at 256 mV/cm for 10 hrs; 8318 MHz at 256 mV/cm for 10 hrs. The yeast cells were conditioned by further culturing in an extract from the digestive tracts of shrimps, jujube juice and hawthorn juice as described in section 5.2, in the presence of a series of two EM fields: 8301 MHz at 256 mV/cm for 15 hours and 8307 MHz at 256 mV/cm for 15 hours. After the last culture period, the yeast cells are either used within 24 hours to make the biological compositions, or dried for storage as described in section 5.3.

The beneficial effect of this third component of yeast cells on animals was tested as follows: The test was conducted with a species of Chinese shrimp, all having a body length of 1.6±0.1 cm. Four tanks were used per test while the total starting weight of shrimp in each tank is within ≦1%. The test was repeated three times, thus involving a total of 12 tanks. The first group of animals (Group A) were fed a diet comprising a mixture of antibiotics as shown in Table 6A, and cultured in water that has been treated with the chemicals in Table 6B.

The animals of Group B were fed a diet comprising activated AS2.982 yeast cells. The activated yeast cells were present in an additive which was prepared by mixing dried cells with zeolite powder (less than 200 mesh) at a ratio of 1×10⁹ yeast cells per gram of zeolite powder. For every 995 kg of basic feed, 5 kg of the additive was added, yielding an additive that comprises 0.5% yeast additive by weight. The third group of animals (Group C) was fed a diet which contains an additive that was prepared identically to that used in Group B except that the AS2.982 yeast cells were not activated. The animals of Group D were fed the basic diet with neither antibiotic nor yeast additives. After five months, the health status of the animals in various groups are shown in Table 9 below.

TABLE 9
Yield of aquatic animals fed with different diets

| Group | Total weight of 3 tanks | % relative to Group A |
|---|---|---|
| A | 66.2 kg | 100 |
| B | 71.9 kg | 108.6 |
| C | 28.3 kg | 42.7 |
| D | 24.8 kg | 37.4 |

To prepare the fourth component, a starting culture containing about 10⁵ cells/ml of AS2.406 is placed into the container (2) as shown in FIG. 1 containing a medium with the composition as shown in Table 4. Initially, the yeast cells are cultured for about 18 hours at 32° C. without an EM field. Then, in the same medium, at 32° C., the yeast cells are cultured in the presence of a series of eight EM fields applied in the order stated: 8426 MHz at 169 mV/cm at 25 hrs; 8432 MHz at 169 mV/cm at 25 hrs; 8438 MHz at 169 mV/cm at 10 hrs; 8443 MHz at 169 mV/cm at 10 hrs; 8426 MHz at 287 mV/cm at 19 hrs; 8432 MHz at 287 mV/cm at 19 hrs; 8438 MHz at 287 mV/cm at 9 hrs; 8443 MHz at 287 mV/cm at 9 hrs. The yeast cells were conditioned by further culturing in an extract from the digestive tracts of shrimps , jujube juice and hawthorn juice as described in section 5.2, in the presence of a series of two EM fields: 8426 MHz at 287 mV/cm for 19 hours and 8432 MHz at 287 mV/cm for 19 hours. After the last culture period, the yeast cells are either used within 24 hours to make the biological compositions, or dried for storage as described in section 5.3.

The beneficial effect of this fourth component of yeast cells on animals was tested as follows: The test was conducted with a species of Chinese shrimp, all having a body length of 1.6±0.1 cm. Four tanks were used per test while the total starting weight of shrimp in each tank is within ≦1%. The test was repeated three times, thus involving a total of 12 tanks. The first group of animals (Group A) were fed a diet comprising a mixture of antibiotics as shown in Table 6A, and cultured in water that has been treated with the chemicals in Table 6B.

The animals of Group B were fed a diet comprising activated AS2.406 yeast cells. The activated yeast cells were present in an additive which was prepared by mixing dried cells with zeolite powder (less than 200 mesh) at a ratio of 1×10⁹ yeast cells per gram of zeolite powder. For every 995 kg of basic feed, 5 kg of the additive was added, yielding an additive that comprises 0.5% yeast additive by weight. The third group of animals (Group C) was fed a diet which contains an additive that was prepared identically to that used in Group B except that the AS2.406 yeast cells were not activated. The animals of Group D were fed the basic diet with neither antibiotic nor yeast additives. After five months, the health status of the animals in various groups are shown in Table 10 below.

TABLE 10
Yield of aquatic animals fed with different diets

| Group | Total weight of 3 tanks | % relative to Group A |
|---|---|---|
| A | 61.7 kg | 100 |
| B | 71.4 kg | 115.7 |
| C | 26.7 kg | 42.3 |
| D | 25.6 kg | 41.5 |

The four above-described preparations of activated yeast cells were conditioned to improve its performance in vivo. Approximately 10 ml of each yeast cell culture (each containing 4×10⁶ cells/ml) were added to 500 ml of the culture medium of Table 5. The mixture (13) is placed in the container (11) as shown in FIG. 2 and cultured in the presence of electromagnetic fields with the frequencies at 7497-7516 MHz, 6800-6820 MHz, 8300-8320 MHz, and 8425-8445 MHz, and a field strength in the range of 260 to 320 mV/ml. The mixture was cultured for 48 hours inside an incubator. The incubation temperature was set to cycle between a minimum of 5° C., room temperature, and a maximum of 35° C. Each cycle takes three hours to complete and is repeated until the 48 hours is up. The mixture of activated yeast cells are separated from the culture medium and stored between 0° C. and 4° C.

A biological feed additive comprising all four yeast cell components was prepared by mixing dried activated cells of each component with zeolite powder (less than 200 mesh) at a ratio of 1×10⁹ yeast cells per gram of zeolite powder. For every 995 kg of basic feed, 5 kg of the yeast and zeolite powder mixture was added, yielding an additive that comprises 0.5% yeast and zeolite powder by weight. The test was conducted with a species of Chinese shrimp, all having a body length of 1.6±0.1 cm. Four tanks were used per test while the total starting weight of shrimp in each tank is within ≦1%. The test was repeated three times, thus involving a total of 12 tanks. The first group of animals (Group A) were fed a diet comprising a mixture of antibiotics as shown in Table 6A, and cultured in water that has been treated with the chemicals in Table 6B.

The animals of Group B were fed a diet comprising the biological feed additives. The third group of animals (Group C) was fed a diet which contains an additive that was prepared identically to that used in Group B except that the yeast cells were not activated. The animals of Group D were fed the basic diet with neither antibiotic nor yeast additives. After five months, the health status of the animals in various groups are shown in Table 10 below.

TABLE 10
Yield of aquatic animals fed with different diets

| Group | Total weight of 3 tanks | % relative to Group A |
|---|---|---|
| A | 64.6 kg | 100 |
| B | 87.9 kg | 136.1 |
| C | 28.4 kg | 43.9 |
| D | 26.1 kg | 40.4 |

The above results indicate that the biological composition of the invention is a valuable animal feed additive that can be used to maintain the health of the animal, and help the animal recover from an infection.

The present invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

What is claimed is:

1. A biological composition comprising at least one of the following yeast cell components:
(a) a first yeast cell component comprising a plurality of yeast cells that are prepared by culturing the yeast cells in an electromagnetic field or a series of electromagnetic fields having a frequency in the range of 7497 to 7516 MHz and a field strength of 3.5 to 200 mV/cm;
(b) a second yeast cell component comprising a plurality of yeast cells that are prepared by culturing the yeast cells in an electromagnetic field or a series of electromagnetic fields having a frequency in the range of 6800 to 6820 MHz and a field strength of 4.5 to 190 mV/cm;
(c) a third yeast cell component comprising a plurality of yeast cells that are prepared by culturing the yeast cells in an electromagnetic field or a series of electromagnetic fields having a frequency in the range of 8300 to 8320 MHz and a field strength of 11 to 290 mV/cm; and
(d) a fourth yeast cell component comprising a plurality of yeast cells that are prepared by culturing the yeast cells in an electromagnetic field or a series of electromagnetic fields having a frequency in the range of 8425 to 8445 MHz and a field strength of 14 to 320 mV/cm.

2. The biological composition of claim 1 which comprises the yeast cell components of (a), (b), (c) and (d).

3. The biological compositions of claim 1 or 2, wherein the yeast cells are cells of Saccharomyces.

4. The biological composition of claim 1 or 2, wherein the yeast cells are cells of Saccharomyces cerevisiae.

5. The biological composition of claim 1 or 2 in which the yeast cells are dried.

6. An animal feed composition comprising the biological composition of claim 1 or 2, and aquaculture feed.

7. The animal feed composition of claim 6 wherein the biological composition further comprises zeolite powder at a ratio of about 10⁹ yeast cells to 1 g of zeolite powder.

8. The animal feed composition of claim 7 in which 0.5% by weight is the biological composition of claim 1 or 2.

9. A method for preparing a biological composition, said method comprising culturing a plurality of yeast cells in an electromagnetic field or a series of electromagnetic fields having a frequency in the range of 7497 to 7516 MHz and a field strength of 3.5 to 200 mV/cm.

10. The method of claim 9, wherein said method further comprises culturing the plurality of yeast cells in one or more of the electromagnetic fields in a culture medium comprising an extract from the digestive tracts of shrimps, wild hawthorn juice, and wild jujube juice.

11. A method for preparing a biological composition, said method comprising culturing a plurality of yeast cells in an electromagnetic field or a series of electromagnetic fields having a frequency in the range of 6800 to 6820 MHz and a field strength of 4.5 to 190 mV/cm.

12. The method of claim 11, wherein said method further comprises culturing the plurality of yeast cells in one or more of the electromagnetic fields in a culture medium comprising an extract from the digestive tracts of shrimps, wild hawthorn juice, and wild jujube juice.

13. A method for preparing a biological composition, said method comprising culturing a plurality of yeast cells in an electromagnetic field or a series of electromagnetic fields having a frequency in the range of 8300 to 8320 MHz and a field strength of 11 to 290 mV/cm.

14. The method of claim 13, wherein said method further comprises culturing the plurality of yeast cells in one or more of the electromagnetic fields in a culture medium comprising an extract from the digestive tracts of shrimps, wild hawthorn juice, and wild jujube juice.

15. A method for preparing a biological composition, said method comprising culturing a plurality of yeast cells in an electromagnetic field or a series of electromagnetic fields having a frequency in the range of 8425 to 8445 MHz and a field strength of 14 to 320 mV/cm.

16. The method of claim 15, wherein said method further comprises culturing the plurality of yeast cells in one or more of the electromagnetic fields in a culture medium comprising an extract from the digestive tracts of shrimps, wild hawthorn juice, and wild jujube juice.

17. A method of making an animal feed composition, said method comprising
(a) culturing one or more of the yeast cell components of claim 1,
(b) drying the yeast cell components of (a), and
(c) mixing the dried yeast cells with zeolite powder and shrimp feed.

18. The method of claim 17, wherein the drying step comprises (i) drying at a temperature not exceeding 65° C. for a period of time such that the yeast cells become dormant; and (b) drying at a temperature not exceeding 70° C. for a period of time to reduce the moisture content to below 5%.

19. A method for reducing the incidence of infectious diseases in a shrimp culture comprising feeding the shrimps for a period of time an animal feed composition comprising at least one of the following yeast cell components:
(a) a first yeast cell component comprising a plurality of yeast cells that are prepared by culturing the yeast cells in an electromagnetic field or a series of electromagnetic fields having a frequency in the range of 7497 to 7516 MHz and a field strength of 3.5 to 200 mV/cm;
(b) a second yeast cell component comprising a plurality of yeast cells that are prepared by culturing the yeast cells in an electromagnetic field or a series of electromagnetic fields having a frequency in the range of 6800 to 6820 MHz and a field strength of 4.5 to 190 mV/cm;
(c) a third yeast cell component comprising a plurality of yeast cells that are prepared by culturing the yeast cells in an electromagnetic field or a series of electromagnetic fields having a frequency in the range of 8300 to 8320 MHz and a field strength of 11 to 290 mV/cm; and
(d) a fourth yeast cell component comprising a plurality of yeast cells that are prepared by culturing the yeast cells in an electromagnetic field or a series of electromagnetic fields having a frequency in the range of 8425 to 8445 MHz and a field strength of 14 to 320 mV/cm.

20. The method of claim 19, wherein the animal feed composition comprises the yeast cell components of (a), (b), (c) and (d), and zeolite powder.

21. The method of claim 19, wherein said yeast cells are Saccharomyces cerevisiae cells.

22. The method of claim 19, wherein the yeast cell components and zeolite powder comprises 0.5% by weight of the animal feed composition.

23. The composition of claim 1 or 2, wherein the plurality of yeast cells used in preparing the first yeast cell component comprise cells of Saccharomyces cerevisiae AS2.502, wherein the plurality of yeast cells used in preparing the second yeast cell component comprise cells of Saccharomyces cerevisiae AS2.562, wherein the plurality of yeast cells used in preparing the third yeast cell component comprise cells of Saccharomyces cerevisiae AS2.982, and wherein the plurality of yeast cells used in preparing the fourth yeast cell component comprise cells of Saccharomyces cerevisiae AS2.406.

24. The animal feed composition of claim 6, wherein the plurality of yeast cells used in preparing the first yeast cell component comprise cells of Saccharomyces cerevisiae AS2.502, wherein the plurality of yeast cells used in preparing the second yeast cell component comprise cells of Saccharomyces cerevisiae AS2.562, wherein the plurality of yeast cells used in preparing the third yeast cell component comprise cells of Saccharomyces cerevisiae AS2.982, and wherein the plurality of yeast cells used in preparing the fourth yeast cell component comprise cells of Saccharomyces cerevisiae AS2.406.
